(19) United States
(12) Patent Application Publication
(10) Pub. No.: US 2007/0298108 A1
(43) Pub. Date: Dec. 27, 2007

(54) Pharmaceutical Formulation

(76) Inventors: Peter Svete, Borovnica (SI); Rok Grahek, Kranj (SI); Vlasta Humar, Stahovica (SI); Breda Husu-Kovacevic, Ljubljana (SI); Zdenka Jerala-Strukelj, Mavcice (SI)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/590,889

(22) PCT Filed: Feb. 28, 2005
(86) PCT No.: PCT/EP05/02108

§ 371 Date: Jun. 4, 2007

(30) Foreign Application Priority Data
Mar. 1, 2004 (SI) P200400067

(51) Int. Cl. A61K 9/36; A61K 31/41; A61P 13/12

(52) U.S. Cl. 424/480; 514/381

(57) ABSTRACT

Composition were developed which stabilize an active pharmaceutical ingredient in polymorph form susceptible to degradation or interconversion into other polymorph forms, where stabilizing substance is conveniently among silicon dioxide, silicified microcrystalline cellulose, magnesium oxide and polyethylene glycol.

### FIELD OF THE INVENTION

The disclosed invention relates to a new method of stabilization of pharmaceutical active ingredients, particularly within pharmaceutical formulations, to prevent degradation and/or conversion of one polymorph form into other polymorph forms.

### BACKGROUND OF THE INVENTION

The tendency to exist in different polymorph forms is known in many classes of active pharmaceutical ingredients. Candesartan, irbesartan, telmisartan, losartan, atorvastatin and pravastatin are just a few examples of active pharmaceutical ingredients known to exist in different polymorph forms. Polymorphs are the forms of the same substance with different space (crystal packing) arrangements which can have different level of order, i.e., crystallinity, where lower crystallinity causes peaks to broaden or even can have on order. The ultimate form of non-orderness of a solid is amorphous state, which does not show the repeatability of molecular directions and positions in a solid. Completely amorphous substance thus shows diffuse dispersion of X-ray radiation, which is substantially manifested in continuum of diffractions throughout the whole of the measured range. Metastable polymorphs (nonequilibrium state of a material with respect to some transition, conversion, or reaction but stabilized kinetically either by rapid cooling or by some molecular characteristics) are also known.

For instance, losartan (2-n-butyl-4-chloro-5-hidroxymethyl-1-[[2′-(1H-tetrazole-5-yl)[1.1′-biphenyl]-4-yl]-1H-imidazole) in the form of its potassium salt exists in at least two anhydrous polymorph forms (form I and II), which interconvert. [Pharm. Res. 10 (1993). 900]. According to WO 03/048135 there is also an amorphous form and a polymorph form having between 12% and 16% of bound water molecules, as well as other forms.

Many active pharmaceutical substances (“actives”) are desirably employed in pharmaceutical formulations in amorphous form. However, if water is absorbed by an amorphous solid this lowers the glass transition temperature of the solid, acts as a plasticizer and increases molecular mobility, leading to an increase in the rate and extent of conversion to other polymorph forms [Chem. Pharm. Bull., 28, 2565-2569 (1980).]

Similar behaviour is observed for other polymorph forms that can under specific conditions—e.g. under environmental influences such as elevated temperature and high relative humidity—convert into another form, which may be more stable under such conditions but can have less advantageous pharmaceutical or pharmacokinetic properties. In particular, spontaneous crystallization or re-crystallization and consequently a change of physical or chemical characteristics can occur under certain storage or manufacturing conditions.

It is consequently desirable to find ways of stabilizing polymorphic forms against adverse environmental influences.

Losartan potassium intended for immediate release is marketed in the form of talc polished coated tablets. To date, the need to stabilize the active ingredient in bulk or in the exposed tablet core has not yet been recognized.

### SUMMARY OF THE INVENTION

In a first aspect, the invention provides a pharmaceutical composition comprising an active pharmaceutical ingredient which exists in a first polymorph form susceptible to degradation or interconversion into one or more other polymorph forms, and further comprising a stabilizing substance selected from the group consisting of colloidal silicon dioxide, finely divided silicon dioxide, silicified microcrystalline cellulose, magnesium oxide and polyethylene glycol, and optionally one or more pharmaceutically acceptable excipients.

Particularly the invention is related to the compositions of potassium salt of losartan, preferably in the amorphous form or in the the polymorph form exhibiting its strongest diffractions in a powder X-ray diffractogram at around 2Θ=6.9, 13.8, 20.6, 24.0, 24.8, 28.7 and 29.2° particularly incorporated into solid dosage form, most particularly where the solid form is a film coated tablet.

In a second aspect, the invention provides a pharmaceutical composition coated with a film coating comprising stearic acid or ethylcellulose, particulary in an amount of from about 0.1% to about 1.7% by weight of the pharmaceutical composition.

In a further aspect, the invention contemplates the use of a composition provided for the manufacture of a medicament. Particularly for treating hypertension and/or chronic renal failure, most particularly where the composition comprises potassium salt of losartan.

In yet another aspect, the invention provides a method of stabilization of an active pharmaceutical ingredient which can exist in more than one polymorph form, and which is susceptible to environmental influences comprising adding finely colloidal silicon dioxide, finely divided silicon dioxide, silicified microcrystalline cellulose, magnesium oxide or polyethylene glycol to said active pharmaceutical ingredient, preferably the stabilization is achieved by adding silicon dioxide.

Possible aspect of the invention is also the use of finely divided silicon dioxide for the stabilization of an active pharmaceutical ingredient which exists in a first polymorph form to prevent the conversion of the active pharmaceutical ingredient to other polymorph forms, preferably in amounts of 1% to 33% by weight relative to the active pharmaceutical ingredient.

### DETAILED DISCLOSURE OF THE INVENTION

We have surprisingly discovered that the amorphous potassium salt of losartan crystallizes under the influence of absorbed water, and different polymorph forms of this substance interconvert in the presence of water. In particular, polymorphs other than form I tend to convert into form I. For example, a polymorph form of losartan potassium exhibiting strongest diffractions in powder X-ray diffractogram (diffraction pattern) at around 2θ=6.9, 13.8, 20.6, 24.0, 24.8, 28.7 and 29.2° (“form X”) interconverts into others polymorph forms. Disclosed 20 values are contemplated to be within ±0.2, preferably ±0.1 of the listed value.

Further, we have succeeded in developing a new method of stabilization of pharmaceutical active ingredients, particularly those susceptible to degradation, against detrimental environmental influences. Most harmful environmental influence is a presence of water. This method can be applied to any active, and in particular to losartan potassium. The active can be stabilized in bulk or contained within a pharmaceutical composition, such as a unit dose pharmaceutical composition (finished dosage form), e.g. in solid form.

Stabilization can be measured by measuring the mass lost on drying and detecting and quantitatively assessing the polymorphs present by suitable technique, for example a suitable thermochemical technique such as DSC or RTG or suitable spectroscopic technique, such as Raman, IR, XRPD, of which XRPD is the most suitable, where one can measure the relative intensities (area) of peaks specific for each specific polymorph. Lost on drying is a parameter that reflects extend of conversion in a substance itself as well as when it is mixed with different excipients that usually have higher water content than drug substance. Stabilization effect of different mixtures is expressed when conversion into another polymorph is inhibited even at higher water content of mixtures or even though they are exposed to the stress conditions of higher humidity. This technique is especially suitable for substances which are extremely sensitive to humidity and converts into another polymorph form even though it is exposed lower e.g. 30-40% relative humidity, such as losartan potassium form X.

The stabilization method of the invention can be applied to stabilize the active for the purposes of storage, handling, and/or transport. The stabilizing substances absorb or chemically bind water molecules fast and strong enough to prevent water absorption to the surface of particles of the active pharmaceutical ingredient. In accordance with the invention any one of the following or mixtures thereof or mixture comprising it can be added to the active as a stabilizing substance: magnesium oxide or calcium oxide or silicon dioxide or polyethylenglycol or croscarmellose sodium. The silicon dioxide can be colloidal silicon dioxide, for example as sold under the trade name Aerosil, or anhydrous silicon dioxide, preferably in the form of a finely divided silicon dioxide, for example as sold under the trade name Syloid or can be in a synergistic intimate physical mixture with another substance. Silicified microcrystalline cellulose, for example as sold under trade name Prosolv is such mixture of two components; microcrystalline cellulose and colloidal silicon dioxide.

Coloidal silicon dioxide is preferably of small particle size and large specific surface area with desirable flow characteristics—those needed to improve the flow properties of dry powders in processes, e.g., tableting. Finely divided silicon dioxide is a preferred stabilizing substance according to the present invention and is an amorphous powder characterized by an internal structure of sponge like pores. Several grades of commercially available Syloid™ are characterized by the porosity, average particle size, and the surface treatment. Preferably finely divided silicon dioxide has small particles bellow 20 μm, fine pores and large specific area above 250 m²/g. The preferred grade Syloid AL-1 has an average particle size 6.0-7.6 μm (Malvern), BET surface area is 750 m²/g and is not surface treated, however silicon dioxide can be treated with an organic coating i. e. with a wax

Similarily as above also croscarmellose sodium which is a crosslinked polymer of carboxymethylcellulose sodium such as sold under the tradename Ac-di-sol or solid grades of polyethyleneglycol for example: PEG 1000-PEG 20000, preferably PEG 4000-PEG 8000 can be used. PEG 6000 is a preferred polyethyleneglycol for use as a stabilizing substance.

In addition to incorporating a stabilizing substance in a pharmaceutical composition with an active, the stability of the active can be further improved by preventing water or moisture penetrating through to the active by forming tablets, and coating the tablets with a coating that is substantially impermeable to water. The optimal coating should nevertheless posses certain physical properties allowing release of drug when ingested.

In a preferred embodiment the pharmaceutical compositions of the invention comprise a coating comprising stearic acid or ethylcellulose, which we have surprisingly found toprovide for the maximum protection of the cores comprising an active pharmaceutical ingredient in a polymorph form that is susceptible of interconversion or crystallization into other forms.

The weight ratio of stearic acid in the coat relative to the weight of whole finished dosage form is ideally from 0.1% to about 1.7%, preferably from 0.2% to 0.9%. most preferably about 0.6%.

The active pharmaceutical ingredient can be any known active which is susceptible to degradation and/or interconversion to other forms under particular environmental conditions. The active is preferably one which can exist in multiple polymorphic forms, and in particular one which converts into one or more other polymorphic forms when exposed to adverse environmental conditions such as high relative humidity or high temperatures. In principle the method of stabilization of the invention can be applied to all active pharmaceutical ingredients which can change crystal structure due to the environmental influences, such as the presence of water. The stabilized substance should be capable of specific chemical interactions with stabilizing substance (e.g. H-bonding, ion-dipole, inclusion complex). For instance, the actives may be a member of the classes of ACE inhibitors, Angiotensin II antagonists, HMG-CoA reductase inhibitors, non-steroidal anti-inflammatory drugs and others. Particular examples of actives useful in practising the invention are: atorvastatin, candesartan, fluvastatin, indomethacin, irbesartan, perindopril, quinapril, pravastatin and losartan.

Above actives can be used for the manufacturing of medicament, which are preferably the finished dosage forms of the pharmaceutical compositions of the invention. Preferably they are solid dosage forms. They compositions of the invention can conveniently be used for the manufacturing of the medicaments for treating hypertension and/or chronic renal failure as in case of ACE inhibitors and Angiotensin II antagonists or can be conveniently used for the manufacturing of the medicaments for treating lipid disordes such as hypercholesterolemia as in case of HMG-CoA reductase inhibitors. Preferably the compositions of the invention, comprising losartan will be used for the manufacturing of a medicament for treating hypertension and/or chronic renal failure.

Preferably, the stabilizing substance is incorporated into the pharmaceutical compositions of the invention in ratios of up to 50%, or up to about 33%, preferably up to 12.5%, preferably from about 1% to about 10%, most preferably in certain embodiments from about 1% to about 3% relative to the weight of the composition.

In a preferred embodiment of the present invention the pharmaceutical composition comprises up to about 10%, such as from about 1% to about 10%, alternatively from about 1% to about 3%, or alternatively from about 3% to about 10% of silicon dioxide, relative to the weight of the composition.

Addition of stabilizing substance not only temporarily stabilizes the active during the time the stabilizing substance binds water, but we surprisingly observed a sustained stabilization even after the mixture had been exposed to 60% relative humidity and the stabilizing substance had already become saturated with water. Thus, the stabilizing substances can prevent the conversion of an active into another polymorphic form under constantly humid conditions, e.g. ambient humidities exceeding 60%, and can prevent the conversion of an active into another polymorphic form in samples with high water content.

Suitable finished dosage form is a drug product that contains a drug substance, generally, but not necessarily in association with one or more ingredients and can be in form of tablets, capsules, pellets, granules, powders, and suppositories or their combined forms.

The finished dosage forms of the pharmaceutical compositions of the invention can be prepared by any suitable method known to the skilled person, for example by direct compression for solid forms. In a particular embodiment a solid unit dosage form comprising losartan potassium is prepared from a mixture of the amorphous form or any polymorph form different from form I, and one or more pharmaceutically acceptable excipients. The dosage form thus formed is then film coated with a coat comprising stearic acid or ethylcellulose.

Silicon dioxide, MgO and CaO can be combined with active as a dry mixture. The mixture is prepared in convenient mixer and sieved through oscillating sieve to give a homogenous dry mixture.

Alternatively the stabilizing substance such as polyethyleneglycol can be combined with an active for example during wet granulation dissolved or dispersed in a suitable solvent such as ethanol to surround every particle of an active with a polymer shield.

To prepare a composition of our invention the active in a first polymorph susceptible to degradation or inter conversion into one or more polymorph form and stabilizing substance such as anhydrous colloidal silicon dioxide or croscarmellose sodium are mixed in container and sieved. In the container one or more suitable fillers, diseintegrants, binders, or any other suitable excipient or another stabilizing substance such as silicified microcrystalline cellulose is added and mixed. Optionally a lubricant such as magnesium stearate is sieved and added into container. Inactive ingredients (excipients) which may function as different fillers, binders, disintegrants, glidants, lubricants, excipients that enhance the absorption of drugs from gastrointestinal tract, and other excipients such as lakes, aromas, colorants, may be incorporated.

To produce the composition in form tablets the final mixture is blended and compressed on a rotary tableting machine.

The compositions of the invention can be film coated as follows: in a suitable solvent such as alchohol film former such as hydroxypropyl cellulose, plasticizer such as triethyl citrate and coating agent such as stearic acid or ethylcellulose are dissolved or suspended. Said suspension is combined with glidant and polishing agent such as talc and optionally coloring agent, lakes or any other commonly used excipients for the coating optionally said excipients being suspended in another or same solvent. Film coating process is performed on a suitable coater and comprises preheating, film coating, drying, cooling and polishing.

In the preferred embodiment a pharmaceutical composition comprising losartan potassium in a first polymorph form susceptible to degradation or interconversion into one or more other polymorph forms losartan, anhydrous colloidal silica, and croscarmellose sodium are mixed in container and sieved on sieve such as Frewitt MG 636 through 0.71 mm. Thereto silicified microcrystalline cellulose is added and mixed for up to 1 hour, preferably 10 minutes. Magnesium stearate is sieved through 0.3 mm sieve and added thereto. The final mixture is blended for up to 15 minutes, preferably 3 minutes. The final dry mixture is compressed into tablets on a rotary tableting machine. For the film coating in dehydrated alchohol while mixing hydroxypropyl cellulose, triethyl citrate and stearic acid are slowly added at speeds of 3000-8000 rpm, during up to one hour, preferably during 15-30 minutes. Thereto a separate suspension of titanium dioxide, ferric oxide and talc in dehydrated alcohol is added and diluted with dehydrated alcohol. Film coating is applied with the suspension flow is 40-100 g/min and air flow is 900-1000 m³/h.

Coatings of different thickness can be applied. Usually a coating up to 10%, preferably from about 3% to about 9%, by weight relative to the core weight is applied.

In a preferred specific embodiment the pharmaceutical composition is an oral finished dosage form comprising active pharmaceutical ingredient in a polymorph form that tends to convert i.e. crystallize into a more stable form, preferably a pharmaceutical composition comprising the potassium salt of losartan in an amount of from 25% to 50% by weight of the composition, most preferably in amount of about 30% by weight, and further comprising from about 50% to about 70% by weight, preferably about 60% by weight of silicified microcrystalline cellulose; from about 2% to about 5% by weight, preferably about 4% of a disintegrant (for example croscamellose sodium); up to about 3%, preferably from 0.1 to 1.5%, most preferably about 0.5% magnesium stearate or any other suitable lubricant and up to about 10%, preferably from about 1% to about 10%, most preferably around 3% of anhydrous silicon dioxide.

The following Examples further illustrate the invention. They are provided for illustrative purposes only, and are not intended to limit the invention in any way.

### EXAMPLES

### Example 1

### Comparative Stability Testing of Binary Mixtures of Losartan Potassium with a Selection of Excipients

Mixtures of the potassium salt of losartan in the polymorph form exhibiting its strongest diffractions in a powder X-ray diffractogram at around 2θ=6.9, 13.8, 20.6, 24.0, 24.8, 28.7 and 29.2 (from hereon API) with different inactive ingredients are tested under the influence of humidity according to the stability study protocol described below, and the results are presented in Table I.

The following inactive ingredients are used: Syloid™ (anhydrous colloidal silicon dioxide), magnesium oxide, PVP K-25 (polivinylpyrolidone), PEG 6000 (polyethyleneglycol), anhydrous lactose, Ac-di-sol (croscarmellose sodium), magnesium stearate, Prosolv (silicified microcrystalline cellulose), Aerosil (anhydrous colloidal silicon dioxide)

Storage conditions: Open dish study—constant influence of humidity Testing mixtures are put into open petri dishes and stored in a thermostatically controlled chamber at a temperature of 25° C. (±2° C.) and 60% (±5%) relative humidity

Storage conditions: Vial study—influence of humidity in a closed container (simulation of packaging)

Testing mixtures are put into vials, exposed to 60% humidity for 18 hours, then closed with rubber stoppers and stored in a thermostatically controlled chamber at temperature of 25° C. (±2° C.) and 60% (±5%) relative humidity.

Duration of study and time points:

Mixtures are tested for up to 5 days in open petri dishes and up to 4 and 9 days in vials, and the following parameters were analysed at intervals of a few days or until the occurrence of significant change: appearance, loss on, conversion of a polymorph form (detected using powder X-ray diffraction spectroscopy and expressed as the amount of crystalline or amorphous phase different from the starting polymorph).

TABLE
Changes occurring in mixtures of potassium salt of losartan in polymorph form
X (API) with different inactive ingredients under the influence of humidity

| | | | Loss on drying | Form convers. |
|---|---|---|---|---|
| Mixture | Time points | Appearance | [%] | [%] |
| API:lactose | Initial state | white powder | 0.06 | 0 |
| 1:1 | vial, 9 days | white powder | 0.17 | 100 |
| API:Ac-di-sol | Initial state | white powder | <0.01 | 0 |
| 1:1 | vial, 9 days | white powder | 5.42 | 0 |
| API:Mg stearate | Initial state | white powder | 1.65 | 0 |
| 1:1 | vial, 9 days | white powder | 1.16 | 100 |
| API:Prosolv | Initial state | white powder | 0.11 | 0 |
| 1:1 | vial, 9 days | white powder | 3.11 | 0 |
| API:Aerosil | Initial state | white powder | 0.36 | 0 |
| | vial, 9 days | white powder | 1.11 | 100* |
| | | | Loss on drying | Form conv. |
| Mixture | Time points | Appearance | [%] | [%] |
| Not stabilized | Initial state | white powder | 0.08 | 0 |
| API | vial, 4 days | white slightly | 0.52 | 5 |
| | | sticky powder | | |
| | open dish, | white slightly | 0.93 | 10 |
| | 5 days | sticky powder | | |
| | vial, 9 days | white slightly | | 100 |
| | | sticky powder | | |
| API:Syloid ™ | Initial state | white powder | 0.21 | 0 |
| 2:1 | vial, 4 days | white powder | 6.7 | 0 |
| | open dish, | white powder | 7.0 | 1-2 |
| | 5 days | | | |
| API:MgO | Initial state | white powder | 0.03 | 0 |
| 2:1 | vial, 4 days | white powder | 0.4 | 1-2 |
| | open dish, | white powder | 1.4 | 2-4 |
| | 5 days | | | |
| API:PEG 6000 | Initial state | white powder | <0.01 | 0 |
| 4:1 | vial, 4 days | white slightly | 2.1 | 0 |
| | | sticky powder | | |
| | open dish, | white sticky | 3.5 | 1-2 |
| | 5 days | powder | | |
| API:PVP K-25 | Initial state | white powder | 1.8 | 0 |
| 4:1 | vial, 4 days | white slightly | 3.9 | 100 |
| | | sticky powder | | |
| | open dish, | white sticky | 4.9 | / |
| | 5 days | powder | | |

*Partial conversion into crystalline Form I and partial into amorphous form

The results demonstrate that in mixtures with Syloid™ and PEG 6000 the API remains in the initial polymorph form after 4 days in vials, and under more demanding conditions, after 5 days at constant 60% relative humidity, not more than 1-2% conversion into form I occur. The stabilisation effect occurs even though water content increases up to 3.5-7%. Surprisingly in an equivalent experiment using PVP K-25 instead of PEG 6000 the initial polymorph completely converts into form I after 4 days in the vial.

Results are very similar for mixtures with MgO where only 1-2% conversion can be detected in vials after 4 days and up to 3% after 5 days in an open petri dish. Upon testing the stability of binary mixtures of API with lactose, Ac-di-sol, Mg Stearate, Prosolv and Aerosil for 9 days in vials, the initial polymorph almost completely converts into form I in mixtures with lactose and Mg stearate, and in mixtures with Aerosil also converts to the amorphous form, but in mixtures with Prosolv and Ac-di-sol no conversion of initial polymorph can be detected.

### Example 2

### Film Coated Tablets Comprising Stabilized Losartan Potassium

A pharmaceutical composition (film coated tablet) is prepared comprising losartan potassium and excipients selected for example based on enhancing flow, and improving compaction behavior. Silicified microcrystalline cellulose is found to have excellent compactibility and achieves high dose loading; croscarmellose sodium is added as a disintegrant. Magnesium stearate is selected in this formulation as an excellent lubricant. The quantity of up to 3%, preferably from 0.5 to 1.0% is necessary in the formulation to make compression feasible. Tablets are film coated.

To prepare tablet cores Losartan potassium and anhydrous colloidal silica are mixed, thensilicified microcrystalline cellulose and croscarmellose sodium are added and homogenised by mixing for 10 minutes. The dry mixture is sieved before magnesium stearate is added and the final mixture is blended for 3 min. The final dry mixture is compressed on a rotary tableting machine. To the cores a coating as follows is applied: Hydroxypropylcellulose and triethyl citrate are dissolved while stirring in ethanol, and then homogenised (Ultraturax, 30 min.). A dispersion of titanium dioxide, ferric oxide red, stearic acid and talc in ethanol is added. The dispersion is sprayed onto cores to obtain a film coating in a weight ratio of about 4.8 wt. % with respect to the core. Tablets are also polished with talc.

| Core | mg/tablet | % of core |
|---|---|---|
| Losartan potassium | 100.00 | 31.25 |
| Silicified mycrocrystalline cellulose | 195.60 | 61.12 |
| Anhydrous colloidal silicon dioxide | 10.00 | 3.13 |
| Croscarmellose sodium | 12.80 | 4.00 |
| Magnesium stearate | 1.60 | 0.50 |
| Total mass | 320.00 | 100.00 |

| coating | mg/tablet | % relative to core |
|---|---|---|
| Hydroxypropylcellulose | 10.90 | 3.24 |
| Stearic acid | 2.10 | 0.62 |
| Triethyl citrate | 0.80 | 0.24 |
| Titanium dioxide | 1.08 | 0.32 |
| Ferric oxide red | 0.02 | 0.01 |
| Talc | 1.10 | 0.33 |
| Total mass of the coat | 16.00 | 4.76 |

1. A pharmaceutical composition comprising an active pharmaceutical ingredient which exists in a first polymorph form susceptible to degradation or interconversion into one or more other polymorph forms, and further comprising a stabilizing substance selected from the group consisting of colloidal silicon dioxide, finely divided silicon dioxide, silicified microcrystalline cellulose, magnesium oxide, polyethylene glycol and croscarmellose sodium, and optionally one or more pharmaceutically acceptable excipients.

2. A pharmaceutical composition according to claim 1, wherein said active pharmaceutical ingredient is the potassium salt of losartan.

3. A pharmaceutical composition according to claim 2 wherein the potassium salt of losartan is in the amorphous form.

4. A pharmaceutical composition according to claim 2 wherein the potassium salt of losartan is in the polymorph form exhibiting its strongest diffractions in a powder X-ray diffractogram at around 2Θ=6.9, 13.8, 20.6, 24.0, 24.8, 28.7 and 29.2°.

5. A pharmaceutical composition according to claim 1 which is in the form of a coated tablet.

6. A pharmaceutical composition according to claim 5 characterized in that it is coated with a film coating comprising stearic acid or ethylcellulose in an amount of from about 0.1% to about 1.7% by weight of the pharmaceutical composition.

7. A pharmaceutical composition according to claim 1 wherein said stabilizing substance is finely divided anhydrous silicon dioxide or polyethylene glycol present in amount of about 1% to about 10% by weight of the composition.

8. A pharmaceutical composition according to claim 7 which is a finished dosage form comprising from about 1% to about 10% by weight of the composition of finely divided silicon dioxide.

9. A pharmaceutical composition according to claim 8 wherein said finely divided silicon dioxide is Syloid™.

10. A pharmaceutical composition according to claim 9 comprising from about 3% to about 10% by weight of the composition of Syloid™.

11-17. (canceled)

18. A method for treating hypertension and/or chronic renal failure comprising administering to a patient in need thereof a pharmaceutical composition comprising an active pharmaceutical ingredient which exists in a first polymorph form susceptible to degradation or interconversion into one or more other polymorph forms, and further comprising a stabilizing substance selected from the group consisting of colloidal silicon dioxide, finely divided silicon dioxide, silicified microcrystalline cellulose, magnesium oxide, polyethylene glycol and croscarmellose sodium, and optionally one or more pharmaceutically acceptable excipients.

19. The method according to claim 18 wherein the active pharmaceutical ingredient is a potassium salt of losartan.
